(19) United States
(12) Patent Application Publication
(10) Pub. No.: US 2023/0030080 A1
(43) Pub. Date: Feb. 2, 2023

(54) METHOD AND DEVICE FOR IDENTIFYING A MOTION PATTERN OF A PERSON

(71) Applicant: novapace GmbH, Darmstadt (DE)

(72) Inventors: Lukas Braisz, Darmstadt (DE); Patrick Scholl, Hirschhorn (DE); Simon Staffa, Bremen (DE)

(73) Assignee: novapace GmbH, Darmstadt (DE)

(21) Appl. No.: 17/433,070

(22) PCT Filed: Mar. 5, 2020
(86) PCT No.: PCT/EP2020/055815

§ 371 Date: Aug. 23, 2021

(30) Foreign Application Priority Data
Mar. 6, 2019 (DE) 10 2019 001 509.6

(51) Int. Cl. A61B 5/11; A61B 5/00

(52) U.S. Cl. CPC A61B 5/1126; A61B 5/6807; A61B 5/7264; A61B 5/7455

(57) ABSTRACT

A device and a method for identifying a motion pattern of a person that allow suitable feedback about an identified motion pattern to be produced in real time, to address the disadvantage that there is no provision for direct feedback to the person in the event of abnormal behavior being detected. This is achieved by virtue of a device for identifying a motion pattern of a person having at least one sensor for capturing motion parameters and at least one evaluation unit, which is couplable to the at least one sensor, for evaluating the motion parameters and for producing a motion pattern.

### CROSS REFERENCE TO RELATED APPLICATIONS

This application is the US National Phase of and claims the benefit of and priority on International Application No. PCT/EP2020/055815 having an international filing date of 5 Mar. 2020, which claims priority on and the benefit of German Patent Application No. 10 2019 001 509.6 having a filing date of 6 Mar. 2019.

### BACKGROUND OF THE INVENTION

### Technical Field

The invention relates to a device for identifying a motion pattern of a person, having at least one sensor for capturing motion parameters, at least one evaluation unit, which is couplable to the at least one sensor, for evaluating the motion parameters and for producing a motion pattern, at least one communication apparatus for transmitting the motion parameters and/or the motion pattern to a remote station and having at least one actuator for generating a signal on the basis of the motion pattern produced. In addition, the invention relates to a corresponding method for identifying a motion pattern of a person, wherein motion parameters are captured by at least one sensor, the motion parameters are evaluated by an evaluation unit coupled to the at least one sensor and are converted into a motion pattern, and the motion pattern produced is taken as a basis for at least one actuator to transmit a stimulus signal to the person.

### Prior Art

It is known practice to ascertain motion patterns or motion profiles of persons by observing said persons, recording said persons using imaging methods, or by using various measuring methods to analyze individual motion parameters. All of these methods initially involve a wide variety of observations being made or data being recorded and subsequently, that is to say after the movement has ended, evaluated. This evaluation can be carried out either by way of an analysis by a specialist or by means of appropriate computer programs.

Investigations such as these take place in competitive or else recreational sport, inter alia, where movements of the various limbs are recorded and analyzed at a later time for different types of sport, such as for example running, cycling or swimming. This analysis is used to optimize movement sequences or flows and hence to improve performance. Furthermore, motion profiles or motion patterns are also produced for determining physical malpositions or incorrect movements. After applicable patterns have been produced and analyzed, measures can then be taken that can lead to correction of the incorrect movement or the poor posture. Regular repetition of the analysis and comparison with previous motion profiles thus allow a wide variety of incorrect movements or deformities to be treated.

Additionally, the analysis of different motion patterns or profiles can lead to certain diseases being identified, in particular early. The progression of certain illnesses can also be identified and analyzed by ascertaining different motion profiles or motion symptoms. These methods therefore allow motion patterns or motion profiles to be identified, compared with known motion patterns and possibly corrected, or countermeasures to be taken.

The aforementioned methods or examples of use for identifying and producing a motion pattern or profile all have the disadvantage that the evaluation and analysis and the taking of appropriate measures or countermeasures take place at intervals of time from one another. After the data have been recorded, said data are gathered and analyzed. Depending on the result of this analysis, a countermeasure is then taken if appropriate. There is no provision or possibility for analysis or evaluation of the patterns in real time. For many of the cited applications, however, in particular countermeasures are found to be useful only as a direct reaction to the captured patterns in real time, that is to say in direct temporal succession to the various motion patterns. The disadvantage that can be identified with the known methods is that there is an absence of suitable or direct feedback to the person when abnormal behavior is detected. There is therefore no learning effect.

### BRIEF SUMMARY OF THE INVENTION

The present invention is based on the object of providing a device and a method for identifying a motion pattern of a person that allow suitable feedback about an identified motion pattern to be produced.

A device for achieving this object is a device for identifying a motion pattern of a person, having at least one sensor for capturing motion parameters, at least one evaluation unit, which is couplable to the at least one sensor, for evaluating the motion parameters and for producing a motion pattern, at least one communication apparatus for transmitting the motion parameters and/or the motion pattern to a remote station and having at least one actuator for generating a signal on the basis of the motion pattern produced. Accordingly, there is provision for a device for identifying a motion pattern of a person to have at least one sensor for capturing motion parameters and at least one evaluation unit, which is couplable to the at least one sensor, for evaluating the motion parameters and for producing a motion pattern. Additionally, the device has at least one communication apparatus for transmitting the motion parameters and/or the motion pattern to a remote station and at least one actuator for generating a signal on the basis of the motion pattern produced. The various motion parameters can be measured movements of the limbs, in particular positions, accelerations, speeds and turning, rotational or tilting movements or maybe forces, in particular ground contact forces. This capture of the motion parameters, the evaluation to produce a motion pattern and the generation of a signal or stimulus signal allow correct movements and postures of the persons to be corrected or trained in everyday life or motion therapy, specifically in real time. This in particular lowers a potential risk of falling for some medical conditions. The direct generation of a signal on the basis of the motion pattern produced provides the person with direct feedback about an incorrect movement of at least one limb. This allows training progress when correcting an incorrect movement or treatment progress to be achieved. Finally, this device can allow better medical care for the affected persons as a result of the individualized identification and feedback. The identification of certain movement patterns of the person that provide signs of a particular illness allows healing to be speeded up or made possible early by virtue of appropriate medication on the basis of the motion pattern produced.

In particular Parkinson's disease patients suffer from movement problems. Changes at neuroanatomic level mean that their gait changes in the form of a shortening of step size, shifting of weight to the front foot and dragging of the foot as it trails. In later stages of the illness, “freezing” occurs, where patients are prevented from initiating steps. Patients can often fall in doing so, owing to this incorrect gait or “freezing”. Use of the device or method according to the invention allows such movement patterns to be identified and the illness-related motion patterns, in particular “freezing”, to be broken by generating an appropriate stimulus signal.

Additionally, the device or method described here can be useful in identifying or treating incorrect loadings or movements of the foot and/or other limbs, such as for example flatfootedness, unbalanced loading/movement of limbs in prosthesis wearers or in stroke patients, and excessively high loadings after for example operations or perhaps other neurological diseases such as multiple sclerosis, polyneuropathy, diabetes or in regard to rehabilitation measures. The method described here also provides athletes with a very efficient way of optimizing movement sequences.

Furthermore, there can be provision according to the invention for the evaluation unit to have an artificial neural network for realtime evaluation of the motion parameters or to be couplable to an artificial neural network. This artificial neural network, or this machine learning or deep learning, provides the person with direct feedback relating to their walking or relating to their motion pattern. To evaluate the ascertained motion parameters, these sensor data are processed in a convolutional neural network. Data obtained beforehand, for example from experiments, are then used by the neural network to distinguish between correct and incorrect motion patterns. If a motion pattern is incorrect, feedback is provided. This feedback is used to notify the person that their movement is not correct and they therefore need to concentrate on performing movements and possibly on an adapted weight distribution. In those suffering from Parkinson's disease, it is usually possible to adapt their gait by concentrating. Should the evaluation of the sensor data detect “freezing”, then appropriate feedback can provide an additional sensory stimulus that assists the person in initiating walking.

Another preferred exemplary embodiment of the present invention can provide for the at least one sensor and/or the at least one actuator to be integrated in a shoe, in particular a sole or an insole, and/or in a knee support and/or a wrist support and/or in another sleeve. This positioning of the sensors or of the actuators on different or multiple limbs allows a very holistic or complete motion pattern of the person to be produced. In most cases, motion patterns of persons are very complex. Motion patterns that are restricted just to the position of the foot or the hip afford only limited opportunity to identify certain malpositions or medical conditions. The capture of different motion parameters on different limbs or body parts allows an extremely complete motion pattern to be produced. The powerful neural network allows appropriate countermeasures or sensory stimuli to be initiated almost instantaneously and a comparison with a “desired motion parameter” to be produced. The evaluation unit with the neural network can be carried directly by the person or can be positioned at any remote station. The sensors or actuators on different limbs can all communicate with one another and among one another wirelessly or by wire, which means that direct feedback can be provided on the basis of the identified motion pattern.

Another advantageous embodiment of the present invention can provide for the at least one actuator, which is preferably a vibrating means, an audible signal means or a visual signal means, to be able to generate a haptic, audible and/or visual signal in real time when specific motion patterns are identified. Different signals can be advantageous depending on the body part to be stimulated. As such, when breaking freezing it can be useful for example to reach the person with a mechanical stimulus directly. Haptic signal means or vibrating means for producing a mechanical stimulus can be applied in the foot region to the medial arch, the lateral arch, the metatarsal head, the calcaneus or the hallux.

Preferably, the at least one sensor can be in the form of an acceleration sensor, in the form of a pressure sensor, in the form of an angle sensor, and/or in the form of a position sensor. These sensors can then ascertain different motion parameters. The choice of the different sensors is dependent on the body part or the limb to which the respective sensor is attached. As such, there can be provision for multiple, preferably different, sensors to be arranged on one and/or on multiple body parts of the person in order to produce a comprehensive record of movement from a multiplicity of motion parameters. There can additionally be provision for the sensors to be read continuously or periodically, cyclically at previously defined intervals of time. The sensor data that are read are then evaluated by the evaluation electronics in real time, and the motion pattern that is then produced or identified is taken as a basis for initiating the actuators to generate a signal. The sensor data can be transmitted by cable or wirelessly (for example by way of a Bluetooth or WLAN connection). The evaluation unit and/or the communication apparatus can be integrated in a smartphone as an app or can be integrated in a separate apparatus worn on the body. There can be provision for the communication apparatus to transmit the motion patterns of the person at regular intervals for storage at a remote station. Additionally, there can be provision for the motion patterns to be transmitted to a treating doctor so that said doctor can analyze the evolutionary process or any changing motion patterns in order to employ further measures if necessary. The transmission of the motion parameters to an appropriate remote station allows the progress or the behavior of the person to be documented over a relatively long period. This documentation allows both the therapy and training and possibly also medication to be individually and specifically adapted and controlled. This allows both the result of therapy and the training for an athlete, for example, to be improved.

In addition, there can be provision according to the invention for the at least one sensor and/or the at least one actuator and/or the evaluation unit and/or the communication apparatus to be able to be supplied with electrical energy by the kinetic energy of the person. This energy harvesting by the individual components means that they are at least extremely energy-independent of external energy stores. Regular charging of storage batteries or the changing of batteries is dispensed with, which is advantageous in particular for elderly people or people without a technical affinity. In particular when said components are positioned on the foot or on the wrist, efficient energy production can be accomplished by converting the kinetic energy of the person into electrical energy.

To supply the at least one sensor and/or the at least one actuator and/or the evaluation unit and/or the communication apparatus with electrical energy, there can be provision for a replaceable battery, a replaceable or permanently installed storage battery and/or a generator that charges an integrated storage battery. To charge the at least one storage battery, there can be provision for said storage battery to be charged by way of a cable or to be put onto a charging pad for wireless charging. This would require the device to be put onto the pad or a charging station or placed at a distance from the latter. The charging station is connected to the power grid by cable and power supply unit. Integrated coils allow wireless power transmission between the charging station and the device, allowing the storage batteries to be charged. This makes uncomplicated and user-friendly charging possible, for example in the shoe rack. In particular wireless charging of the storage battery by way of a charging pad by transmitting electromagnetic waves is very advantageous for elderly people, since there is no need to connect a charger to a socket or to handle cables. As an alternative to externally chargeable storage batteries or by way of assistance, an integrated generator can convert kinetic energy into electrical energy. It is possible to make use of the dynamo effect for this, for example. The generation of electrical energy by converting kinetic energy is in particular advantageous for sensors, etc., that are attached to body parts that move regularly and a lot, such as for example the feet or legs.

A method for achieving the object cited at the outset is a method for identifying a motion pattern of a person, wherein motion parameters are captured by at least one sensor, the motion parameters are evaluated by an evaluation unit coupled to the at least one sensor and are converted into a motion pattern, and the motion pattern produced is taken as a basis for at least one actuator to transmit a stimulus signal to the person. Accordingly, there is provision for a method for identifying a motion pattern of a person, wherein motion parameters are captured by at least one sensor, the motion parameters are evaluated by an evaluation unit coupled to the at least one sensor and are converted into a motion pattern. The motion pattern produced is taken as a basis for at least one actuator to transmit a stimulus signal to the person.

Furthermore, the method according to the invention can provide for motion patterns to be produced from the captured motion parameters by an artificial neural network in real time. This neural network allows sensor values or measurement data of multiple, preferably different, sensors that are arranged on one and/or multiple body parts of the person to produce a comprehensive motion pattern from a multiplicity of motion parameters in real time. The motion pattern produced or identified is then taken as a basis for the at least one actuator to generate a haptic and/or audible and/or visual signal.

A communication apparatus can then transmit the identified motion pattern to an analysis apparatus for an evaluation, in particular for an evaluation of a change over time. This allows progress or another change in the movement behavior of the person to be ascertained by means of a long-term evaluation of the motion parameters.

Preferably, there can additionally be provision for a motion pattern produced to be compared with a previously classified motion pattern in real time and for a divergence or a concordance between the produced and classified motion patterns to result in the at least one actuator transmitting a stimulus signal to the person in real time.

### BRIEF DESCRIPTION OF THE DRAWINGS

Preferred exemplary embodiments of the present invention are explained in more detail below with reference to the drawing, in which:

FIG. 1 shows a depiction of an exemplary embodiment of a device according to the invention;

FIG. 2 shows a depiction of another exemplary embodiment of the device;

FIG. 3 shows a depiction of another exemplary embodiment of the device;

FIG. 4 shows a depiction of another exemplary embodiment of the device; and

FIG. 5 shows a depiction of another exemplary embodiment of the device.

### DETAILED DESCRIPTION OF PREFERRED EMBODIMENTS

To identify a motion pattern of a person, a device according to the invention has at least one sensor for capturing motion parameters, at least one evaluation unit, which is couplable to the at least one sensor, for evaluating the motion parameters and for producing a motion pattern, and at least one communication apparatus for transmitting the motion parameters and/or the motion pattern to a remote station. Additionally, the device has at least one actuator for generating a signal on the basis of the motion pattern produced. Multiple exemplary embodiments of such a device are depicted below. However, it will be expressly pointed out that these embodiments are only examples and the device according to the invention is not restricted to these exemplary embodiments. On the contrary, there is provision for the device according to the invention to be realizable by a multiplicity of further embodiments.

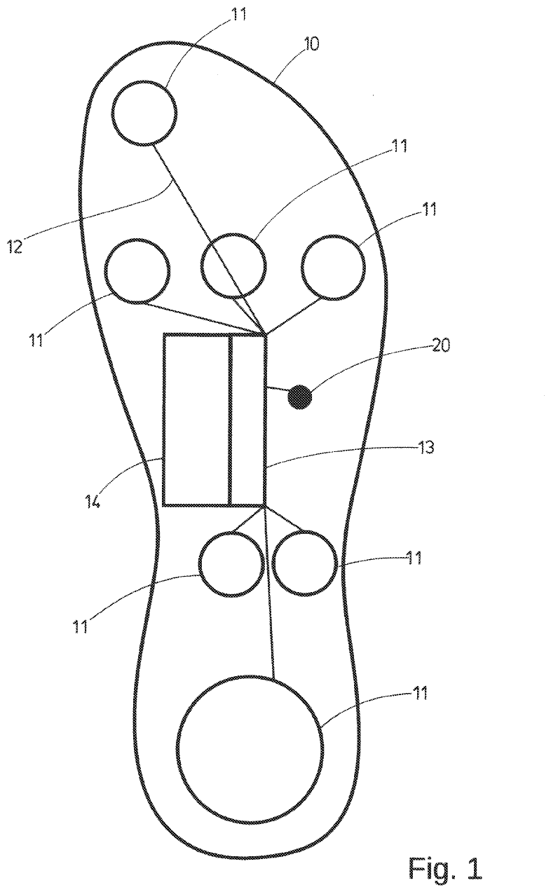

FIG. 1 depicts a sole 10 for a shoe. This sole 10 can also be an insole. In the exemplary embodiment depicted in FIG. 1, the device has seven sensors 11 distributed over the whole surface of the sole 10. The sensors 11 are distributed over the sole 10 in such a way that they produce as complete a representation of the movement of the person as possible. These sensors 11 can be pressure sensors, acceleration or motion sensors, angle sensors, inertial sensors, position sensors or else temperature sensors.

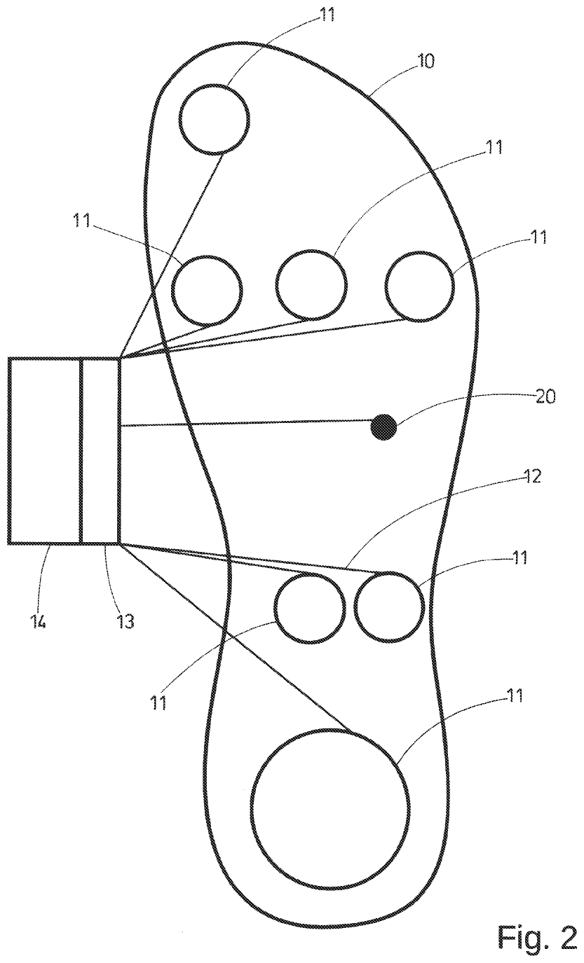

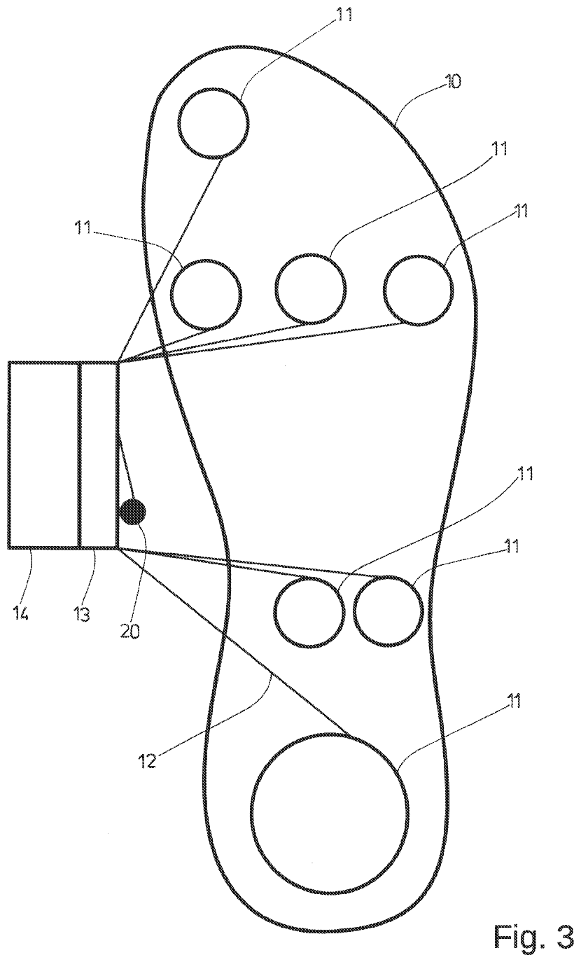

Additionally or instead of at least one sensor 11 from FIG. 1, it is also conceivable for said sensor to be in the form of an actuator for generating a signal. This signal can be an audible, visual or haptic signal. A haptic, that is to say vibratory, signal being preferred in particular when the actuator is positioned on or in the sole 10. As an example of an actuator, FIGS. 1 to 3 depict an actuator 20, which can in particular be a vibrating actuator. Preferably, the actuator 20 is placed where the person is particularly sensitive to sensory stimuli. In general, however, the actuator 20 can be placed at any position (FIGS. 1 and 2), for example even at a different location than the sole, e.g., on the instep (not depicted, see FIG. 3). The actuator 20 can be connected to a control unit 13 and obtain its electrical energy from the battery 14.

The sensors 11 depicted in FIG. 1 are each connected to a unit 13 via electrical lines 12. This unit 13 can comprise both an evaluation unit and a communication apparatus. In particular for evaluating the captured measured values, the evaluation unit has an artificial neural network that can analyze the data in real time and can generate a signal if necessary. The communication apparatus can then be used to transmit the recorded data or the identified or evaluated motion patterns to a remote station. This remote station can be either a smartphone or else a server, which is for example located in a doctor's practice and stores all of the motion patterns of the person for the further analysis.

In addition, the device depicted in FIG. 1 has an energy store or a battery 14. This battery 14 can also be a storage battery. Exemplary embodiments are conceivable in which the storage battery is recharged with electrical energy by the kinetic motion of the person or of the foot. This energy harvesting means that the device or the sensors 11, the actuators and the unit 13 can be operated in an at least almost energy-independent manner.

The sensors 11, possibly actuators 20, and the unit 13 and the battery 14 depicted in FIG. 1 can be arranged on the sole 10 or integrated in the sole. It is additionally possible for said components to be arranged at a different position in a shoe (FIG. 3). As such, it is conceivable for some sensors 11 or actuators 20 also to be arranged on the instep or in the heel region of the foot.

FIG. 2 depicts another exemplary embodiment of the invention. In contrast to the exemplary embodiment depicted in FIG. 1, the unit 13 together with the battery 14 in the exemplary embodiment depicted in FIG. 2 are not integrated in the sole 10 but rather situated at a different location. It is also conceivable for the unit 13 to be located in a handheld device or a smartphone or to be associated with an external device that the person can strap to their belt, for example. The connection between the sensors 11, the actuator 20 and the unit 13 can be made by means of a cable 15 or wirelessly, e.g., by way of Bluetooth or WLAN.

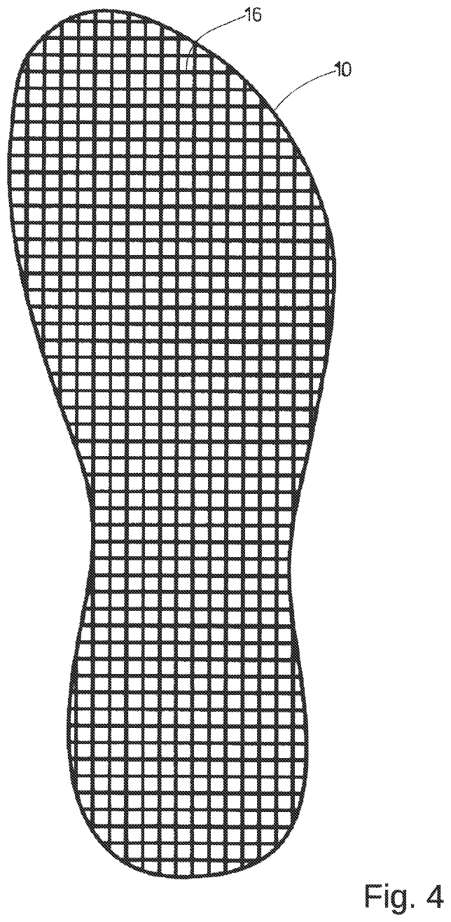

Instead of what is depicted in FIGS. 1, 2 and 3, the sole 10 can also have an associated sensor matrix 16 (FIG. 4). This sensor matrix 16 is distributed over the whole surface of the sole 10 and therefore has a maximum sensitivity. This sensor matrix 16 allows a pressure distribution of the foot on the sole 10 to be ascertained very accurately, for example. Otherwise, the exemplary embodiment depicted in FIG. 4 has the same features as the exemplary embodiments of FIGS. 1 to 3. The sensor matrix 16 can also be used to ascertain other physical properties, such as speed, acceleration or location of the person.

Even though FIGS. 1 to 4 depict only one sole 10, it is conceivable for the device along with said components to be integrated in both soles of the persons. By capturing the motion parameters of both feet, it is possible to produce a very complete motion pattern of the person. Furthermore, it is conceivable for the same sensor arrangement and actuator arrangement as depicted in FIGS. 1 to 4 also to be assembled in appropriate devices for other body parts, such as for example hands, hips, shoulders and the like.

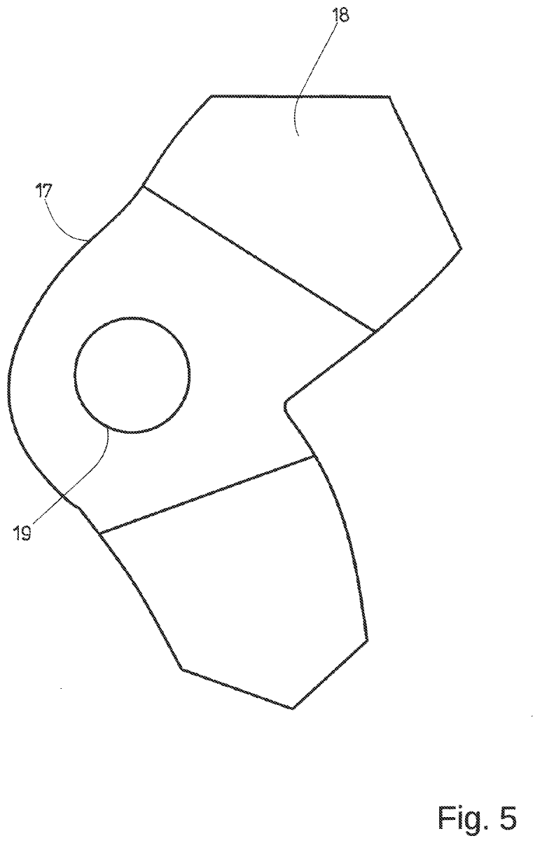

For example, FIG. 5 depicts a knee support 17 that can be pulled over a leg 18 of the person. This knee support 17 has at least one sensor means 19 at the side. This knee support 17 can equally have a unit 13 and an actuator, as depicted in FIGS. 1 to 3. It is therefore also possible for the data captured by the sensor means 19 to be processed and, if necessary, conveyed to a remote station. It is also conceivable for a similar sleeve to be associated with the elbow or the ankle, specifically also comprising an inertial sensor system and/or a sensor matrix. An appropriate device can also convert the kinetic energy of the person at the knee into electrical energy for supplying power to the individual components.

### LIST OF REFERENCE SIGNS

10 sole
11 sensor
12 line
13 unit
14 battery
15 cable
16 sensor matrix
17 knee support
18 leg
19 sensor means
20 vibrating actuator

1. A device for identifying a motion pattern of a person, having at least one sensor (11, 19) for capturing motion parameters, at least one evaluation unit, which is couplable to the at least one sensor (11, 19), for evaluating the motion parameters and for producing a motion pattern, at least one communication apparatus for transmitting the motion parameters and/or the motion pattern to a remote station, and at least one actuator for generating a signal on the basis of the motion pattern produced.

2. The device for identifying a motion pattern as claimed in claim 1, wherein the evaluation unit has an artificial neural network for realtime evaluation of the motion parameters or is couplable to an artificial neural network.

3. The device for identifying a motion pattern as claimed in claim 1, wherein the at least one sensor (11, 19) and/or the at least one actuator is integrated in a shoe, in particular a sole (10) or an insole, and/or in a knee support (17) and/or a wrist support and/or in another sleeve.

4. The device for identifying a motion pattern as claimed in claim 1, wherein the at least one actuator can generate a haptic, audible, and/or visual signal in real time when specific motion patterns are identified.

5. The device for identifying a motion pattern as claimed in claim 1, wherein the at least one sensor (11, 19) is in the form of an acceleration sensor, a pressure sensor, an angle sensor, or a position sensor.

6. The device for identifying a motion pattern as claimed in claim 1, wherein a plurality of the sensors (11, 19) are arranged on one and/or on multiple body parts of the person in order to produce a comprehensive motion pattern from a multiplicity of motion parameters.

7. The device for identifying a motion pattern as claimed in claim 1, wherein the at least one sensor (11, 19) and/or the at least one actuator and/or the evaluation unit and/or the communication apparatus can be supplied with electrical energy by the kinetic energy of the person.

8. A method for identifying a motion pattern of a person, comprising capturing motion parameters by at least one sensor (11, 19), evaluating the motion parameters by an evaluation unit coupled to the at least one sensor (11, 19), converting the evaluated motion parameters into a motion pattern, and using the motion pattern produced as a basis for at least one actuator to transmit a stimulus signal to the person.

9. The method for identifying a motion pattern as claimed in claim 8, wherein the motion pattern is produced from the captured motion parameters by an artificial neural network in real time.

10. The method for identifying a motion pattern as claimed in claim 8, further comprising using multiple sensors (11, 19) that are arranged on one and/or multiple body parts of the person to produce a comprehensive motion pattern from a multiplicity of motion parameters in real time and using the motion pattern as a basis for the at least one actuator to generate a haptic and/or audible and/or visual signal.

11. The method for identifying a motion pattern as claimed in claim 8, further comprising carrying out an evaluation of the motion pattern by virtue of the motion pattern being transmitted to an analysis apparatus by way of a communication apparatus (13).

12. The method for identifying a motion pattern as claimed in claim 8, further comprising comparing a motion pattern produced with a previously classified motion pattern in real time and in which a divergence or a concordance between the produced and classified motion patterns results in the at least one actuator transmitting a stimulus signal to the person in real time.

13. The method for identifying a motion pattern as claimed in claim 10, wherein the multiple sensors (11, 19) are different from each other.

14. The method for identifying a motion pattern as claimed in claim 11, wherein the evaluation is of a change over time.

15. The device for identifying a motion pattern as claimed in claim 4, wherein the at least one actuator is selected from the group consisting of a vibrating means, an audible signal means, and a visual signal means.

16. The device for identifying a motion pattern as claimed in claim 6, wherein the plurality of sensors (11, 19) are different from each other.
